(19) United States
(12) Patent Application Publication
(10) Pub. No.: US 2026/0015236 A1
(43) Pub. Date: Jan. 15, 2026

(54) USES OF A CARBON PRODUCED FROM A METHOD FOR THE MATERIAL TREATMENT OF RAW MATERIALS

(71) Applicant: Merenas Trust Reg., Vaduz (LI)

(72) Inventor: Karl-Heinz HEMMERLE, Vaduz (LI)

(21) Appl. No.: 18/992,281

(22) PCT Filed: Jul. 7, 2023
(86) PCT No.: PCT/EP2023/068838

§ 371 Date: Jan. 8, 2025

(30) Foreign Application Priority Data
Jul. 8, 2022 (EP) 22183760.2

(51) Int. Cl. C01B 32/15; A01C 21/00; A61K 33/44; A61K 47/02; B01D 39/20; B01D 53/04; B01J 20/20; B01J 20/28; C02F 1/28; C02F 1/40; C02F 101/12; C02F 101/32; C05G 5/10; C09K 17/02; C10B 53/02; F16L 59/02; H01G 11/36

(52) U.S. Cl. CPC C01B 32/15; A01C 21/00; A61K 33/44; A61K 47/02; B01D 39/2055; B01D 53/04; B01J 20/205; B01J 20/28066; C02F 1/283; C02F 1/40; C05G 5/10; C09K 17/02; C10B 53/02; F16L 59/028; H01G 11/36; B01D 2253/102; B01D 2253/304; B01D 2253/306; C01P 2002/02; C01P 2004/50; C01P 2004/64; C01P 2006/12; C01P 2006/32; C02F 2101/12; C02F 2101/32

(57) ABSTRACT

An amorphous carbon has a three-dimensional arrangement of carbon nanoparticles as agglomerates and is produced from carbon-containing raw materials by a charring and distillation process. The carbon is suitable for medical use, as thermal and/or refractory and/or radiation-resistant insulating material, as a filter element, as a storage element or for the production of plant products or for planting water-poor regions. In another aspect, an electric energy storage device in the form of a double-layer capacitor has a symmetrical structure, with a housing and a collector each from the outside inwards, with an electrode formed as a carbon layer and a separator with an electrolyte, wherein the carbon layer is formed as agglomerates from an amorphous carbon produced by the method for the material treatment of carbon-containing raw materials and having the structure of the three dimensional arrangement of carbon nanoparticles.

### CROSS-REFERENCE TO RELATED APPLICATIONS

This application is a National Stage Application under 35 U.S.C. § 371 (a) of PCT/EP2023/068838, filed on Jul. 7, 2023, which claims benefit of and priority to European Patent Application No. 22183760.2 filed on Jul. 8, 2022. The entire contents of the foregoing applications are incorporated by reference herein.

The invention relates to uses of a carbon produced from a method based on a charring and distillation process for the material treatment of raw materials.

Devices and methods known from the prior art are provided for the industrial treatment, in particular of waste rubber products, rubber products or rubber-like composite products, such as used tyres, steel rope-reinforced rubber belts, rubberised chain links and conveyor belts, as well as of crushed end-of-life vehicles, organic renewable raw materials, such as wood, contaminated carbon and contaminated soils. Thus, light oil, gas, metals, in particular steel, and carbon are obtained. Conventional plants are based, for example, on the use of rotary kilns, fluidised-bed reactors and drums and processed compressed starting material in a chemically inert atmosphere with the exclusion of oxygen.

DE 199 30 071 C2 describes a method and a device for the recovery of organic substances and substance mixtures. The organic material is brought into contact with fluidised bed material of the combustion fluidised bed. The method produces final products in the form of gases with condensable substances and carbon-containing residues.

DE 39 32 803 A1 discloses a reaction method of organic materials with the addition of boric acid/boron oxide and organic nitrogen compounds in a non-oxidising atmosphere to carbon and graphite.

The operation of conventional plants requires increased expenditure on materials, energy and logistics. Thus, for example, the production of a fluidised bed in fluidised bed reactors causes an increased energy expenditure, since on the one hand the fluidised bed must be produced and maintained and on the other hand the materials to be utilised must be worked up mechanically in such a way that they effectively contact the fluidised bed. High energy costs also arise due to the crushing or compaction of the starting materials in the preparation and during the recycling procedure.

WO 2007/053088 A1 describes a method and a device for treating materials from hydrocarbons. The materials are fed into an inner container, which in turn can be arranged in an outer container. Both containers are each closed with a cover element. The hydrocarbon material is heated by means of microwaves or high-frequency irradiation. The resulting exhaust gases are discharged from the containers through a gas outlet. Two or more containers can be operated in parallel and connected to a gas cleaning plant to maintain a nearly continuous gas flow through the gas cleaning plant.

WO 2010/012275 A2 discloses a device for treating materials with a cylindrical furnace and a control of the process. The inner surfaces of the furnace are provided with an insulating layer of an inorganic heat insulating material. Heating elements are arranged at or on the inner surfaces of the insulating layers. The control of the process by controlling the temperature of the heating elements serves to achieve a high yield of carbon, oil and fuel gas.

DE 10 2012 109 874 A1 shows a device for the material treatment of raw materials with a heating system, a distillation unit and a reaction unit which can be charged with the raw materials as well as a method for operating such a device. The heating system, which can be opened and closed to be charged with the reaction unit, has a head element and a jacket element which is firmly connected to the head element, as well as support elements. The head element is connected to the support elements which can be varied in length in the vertical direction in such a way that by changing the length of the support elements between two end positions, the heating system is opened and closed in the vertical movement direction.

WO 2010/117392 A1 describes different embodiments and methods for producing exposed carbon nanotubes. The methods comprise the steps of suspending carbon nanotubes in a solution which contains a nanocrystalline material, precipitating the exposed carbon nanotubes from the solution and insulating the exposed carbon nanotubes. The methods can further include the steps of producing a solution of carbon nanotubes in an acid and filtering the solution through a filter in order to collect exposed carbon nanotubes with the filter.

KR 2013 0027690 A shows a method for producing carbon nanofibres or a carbon nanofibre string. Carbon nanofibres, which were created by electrospinning a carbon fibre polymer, are laminated and then slit in regular intervals in order to form a twist. Afterwards, the carbon nanofibres are oxidised.

It is an object of the present invention to provide a carbon produced by a method for the material treatment of raw materials, in particular different waste rubber products, such as used tyres and rubber composite products or rubber-like composite products, renewable raw materials, such as wood, shells or fruits, electronic scrap, such as computers and mobile phones, motor vehicles and storage media, such as batteries, with advantageous material properties differing from conventional carbons, for various uses.

The solution to the object of the invention is uses of carbon with a structure of a three-dimensional arrangement of carbon nanoparticles as agglomerates which is produced by a method for the material treatment of carbon-containing raw materials. The carbon is amorphous and the carbon nanoparticles are cross-linked without long-range order, do not exhibit any large-scale graphitic arrangement or structural similarity to graphene and are not arranged as nanotubes. The method has the following steps:
heating a reaction unit charged with raw materials and arranged in a closed heating system and starting a charring and distillation process, wherein the charring and distillation process is carried out by selective heating at a substantially constant temperature within the reaction unit,
removing any developing gases from the reaction unit to a distillation unit by means of an exhaust gas line formed between the reaction unit and the distillation unit and determining the temperature of the gas flowing through the exhaust gas line,
cooling and condensing the gases in the distillation unit, wherein the temperature of the gases is controlled by forced cooling of a cooling section of the distillation unit by means of a heat output dissipated by the gases; and
extracting non-condensable gases, wherein a negative pressure is generated towards the environment within the reaction unit and oxygen is removed from the reaction unit,
cooling the reaction unit and
removing the final products from the reaction unit.

According to the invention, the carbon serves for medical use, as a thermal and/or refractory insulating material, as a filter element, as a storage element or for producing plant products or for planting water-poor regions.

The method is advantageously based on the operation of a device for the material treatment of raw materials. The device has a heating system, a distillation unit and a reaction unit as well as a control device. The reaction unit can be charged with the raw materials. The heating system can be opened and closed to be fitted with the reaction unit. An exhaust gas line for discharging the exhaust gases from the reaction unit is formed between the reaction unit or the heating system and the distillation unit. The distillation unit has a cooling section.

Temperature sensors are formed in the region of the heating system and the distillation unit. In addition, the cooling section of the distillation unit has a device for forced cooling. The device for the forced cooling of the cooling section makes it possible for the cooling section, in comparison, for example, to free convention, to be subjected to a specific flow of a heat carrier fluid—in particular gaseous or liquid—for the removal of heat, or for the flow of heat to flow around it.

The device for the material treatment of raw materials has an extraction device for extracting gases from the reaction unit and generating a negative pressure within the reaction unit. The negative pressure refers to the pressure of the surroundings of the device. The extraction device can be formed as a pump, in particular as a diaphragm pump.

The temperature sensors and the extraction device are connected to the control device.

The device preferably has at least two temperature sensors for determining the temperature within the reaction unit, which, when the heating system is closed, are arranged in an intermediate space formed between the reaction unit and a jacket element of the heating system.

An exhaust gas line formed between the heating system and the distillation unit can have a heating device for heating the exhaust gas line. The heating device, which preferably completely surrounds the exhaust gas line and is advantageously electrically operated, is connected to the control device.

At least one temperature sensor for determining the temperature of exhaust gases discharged from the heating system is preferably provided on the exhaust gas line formed between the heating system and the distillation unit.

A connecting element for connection to a device for introducing a gaseous flushing medium, in particular into the reaction unit, can be provided on the exhaust gas line formed between the heating system and the distillation unit. The flushing medium, for example nitrogen, serves for rendering inert within the reaction unit, reduces the risk of explosion and, as a carrier gas, supports the separation of final products produced during operation of the device.

The cooling section of the distillation unit is advantageously arranged inside an air guide housing. Fans are provided inside a wall of the air guide housing for the targeted conduction of ambient air over the cooling section. The air guide housing with the fans is formed as a device for forcibly cooling the cooling section of the distillation unit with ambient air. The fans are connected to the control device.

The fans, which are formed inside the wall of the air guide housing of the cooling section of the distillation unit, for the targeted conduction of ambient air over the cooling section are preferably arranged on an upper side, in particular on an end face pointing upwards in the vertical direction, or on a side face of the air guide housing.

Alternatively, the cooling section can be formed from at least one double-walled coaxial tube as a device for forced cooling of the cooling section for passing gases in the interior of an inner tube and for passing a heat carrier fluid in the intermediate space between the outside of the inner tube and the inside of the outer tube. The heat carrier fluid is preferably in the liquid state of aggregation and is in particular water or glycol.

An advantage of the device for the material treatment of raw materials is that the extraction device for extracting gases from the reaction unit and generating a negative pressure within the reaction unit is arranged downstream of an oil tank arranged downstream of the distillation unit in the flow direction of the gases. Thus, the negative pressure is also generated within the distillation unit.

The heating system can have a head element and the jacket element, which is firmly connected to the head element, as well as support elements. The head element is arranged mounted on the support elements which can be varied in length in the vertical direction. By changing the length of the support elements between two end positions, the heating system is opened and closed in the vertical movement direction.

The heating system preferably has two support elements which are preferably arranged on both sides of the heating system. According to a first alternative, the support elements are driven by electric spindles. According to a second alternative, the support elements are formed as hydraulic supports.

According to a further development of the device, the jacket element is formed with a hollow cylindrical wall. The wall is open downwards in the vertical direction and closed at the top with a circular hood. The jacket element is connected to the head element at the hood to form a unit.

The jacket element advantageously has heating elements distributed uniformly on the circumference of the inner surface of the wall. To prevent heat transfer to the outside, the wall is formed with a heat insulation made of ceramic powder.

The hood can be formed at the centre point with an exhaust gas port as a connection to an exhaust gas line of the heating system. The exhaust gas line extends from the exhaust port through the hood into the head element of the heating system.

The exhaust gas line advantageously has a connecting element at the distal end to the exhaust gas port of the hood as a connection to the exhaust gas line of the distillation unit.

The gas exhaust line extending from the exhaust gas port through the hood into the head element of the heating system can be formed in the region of the exhaust gas port for compensating thermal expansions with a pipe connection which is automatically variable in length, in particular in the vertical direction.

A further advantage of the device is that the reaction unit is formed with a wall in the form of a hollow cylindrical vessel which is closed at the bottom. The open side of the wall can be closed by means of a cover element.

A high-temperature-resistant seal is advantageously arranged between the wall and the cover element.

The cover element of the reaction unit is preferably formed to be circular and has the exhaust gas port at the centre point. It is particularly advantageous that the exhaust gas port of the cover element and the exhaust gas port of the jacket element engage in one another in the closed state of the heating system and form a tight connection to the exhaust gas line.

The cover element of the reaction unit can be formed with a connecting port for connection to a device for introducing a gaseous flushing medium, in particular nitrogen, into the reaction unit.

The reaction unit can have screen elements inside, which are preferably aligned horizontally and arranged at different heights, at a distance from one another. The screen elements preferably cover the entire cross-section of the reaction unit.

The control device of the device for the material treatment of raw materials serves to control a method for operating the device and, in addition to the temperature sensors, the device for forced cooling, in particular the conveying devices, such as the fans for directing ambient air in a targeted manner over the cooling section or at least one pump for conveying the liquid heat transfer fluid, and the extraction device, is advantageously also connected to a drive of the support elements, a filling level sensor of the oil tank, a pressure sensor and valves of heating circuits of the heating system. The filling level sensor of the oil tank can be formed as a float. The pressure sensor is advantageously arranged in the region of the oil tank. The control device can also be connected to an oil conveying device for extracting the oil of the oil tank, in particular a piston pump. The oil conveying device is put into operation upon a signal sent by the filling level sensor of the oil tank to the control device, and oil is conveyed out of the oil tank.

The method for the material treatment of raw materials can be based on the operation of the described device for the material treatment of raw materials. The method has the following steps:
charging a reaction unit with raw materials,
preheating the reaction unit,
opening a heating system and introducing the reaction unit into the heating system, in particular onto a bottom element of the heating system,
closing the heating system so that the reaction unit is arranged in a closed space,
heating the reaction unit and starting a charring and distillation process, wherein the charring and distillation process is carried out by selective heating at a substantially constant reaction temperature within the reaction unit, wherein the temperature is determined,
removing any developing gases from the reaction unit to a distillation unit by means of an exhaust gas line formed between the reaction unit and the distillation unit and determining the temperature of the gas flowing through the exhaust gas line,
cooling and condensing the gases in the distillation unit, wherein the temperature of the gases is controlled by forced cooling of a cooling section of the distillation unit by means of a heat output dissipated by the gases,
introducing the distillation products into an oil tank and discharging oil,
extracting non-condensable gases from the oil tank, wherein a negative pressure to the environment is generated within the reaction unit and oxygen is removed from the reaction unit,
opening the heating system and removing the reaction unit from the heating system,
cooling the reaction unit, removing the final products from the reaction unit and separating the final products, and
removing the final products from the oil tank.

Targeted heating is understood to mean that the reaction unit arranged within the heating system is heated during the charring and distillation process in such a way that the reaction temperature within the reaction unit, also referred to as the process temperature, is substantially constant and varies only within a predetermined temperature band. In doing so, the reaction temperature is permanently monitored. The value of the temperature is transmitted to the control device, which controls the opening and closing of the valves of heating circuits of the heating system and thus a firing in accordance with a predetermined desired value of the temperature.

When the heating system is closed, an exhaust gas port of the reaction unit is preferably coupled to an exhaust gas port of an exhaust gas line of the heating system, and the exhaust gas line of the heating system and an exhaust gas line of the distillation unit are coupled to one another on a connecting element, so that a gas-tight connection is produced from the reaction unit to the distillation unit. The heating system is advantageously opened and closed by extending and retracting support elements.

With the extraction of non-condensable gases from the oil tank and thus the generation of the negative pressure, the absolute value of the pressure within the reaction unit can be set in the range from 2 mbar to 10 mbar, in particular from about 4 mbar.

In order to cool and condense the gases in the distillation unit, ambient air can be passed in a targeted manner over the cooling section of the distillation unit, or a liquid heat transfer fluid, in particular water as coolant, can flow through the cooling section.

During the procedure of cooling and condensing the gases, the temperature of the gases in the distillation unit is set to a value in a range from 95° C. to 125° C., for example via a volume flow of the ambient air or an output of the fans or a mass flow of a heat transfer fluid. The volume flow of the ambient air or the mass flow of the heat transfer fluid ensures the heat to be dissipated from the cooling section and cools the cooling section. The temperature of the gases is determined in the exhaust gas line formed between the heating system and the distillation unit, in particular the at least one temperature sensor for determining the temperature of exhaust gases discharged from the heating system.

An advantage of the method is that during the charring and distillation process, an exhaust gas line formed between the reaction unit and the distillation unit is heated, in particular to a temperature in the range from 120° C. to 160° C., in particular in order to avoid premature condensation of the exhaust gas before entry into the distillation unit and consequently clogging of the exhaust gas line.

The reaction unit is preferably removed from the heating system at a temperature of the gas flowing through the exhaust gas line of about 60° C.

During the charring and distillation process or during the procedure of cooling the reaction unit, a gaseous flushing medium, in particular nitrogen, is introduced into the reaction unit.

The flushing preferably takes place in each case at time intervals, in particular in order to remove relatively high molecular weight gases from the reaction unit. Flushing by means of inert gas, such as nitrogen, removes undesirable components, such as polyaromatic constituents of polybutadiene or plasticisers, from the reaction unit, in particular during the charring and distillation process. Extraction of non-condensable gases and thus the generation of the negative pressure within the reaction unit and the inflow of the flushing medium into the reaction unit advantageously take place offset in time with respect to one another. Specifically during the procedure of cooling the reaction unit, the flushing medium can be periodically introduced into the reaction unit for a respective duration in the range of two to three minutes.

After the reaction unit has cooled, the reaction unit is preferably opened at a temperature inside the reaction unit in the range from 20° C. to 60° C., in particular in the range from 30° C. to 60° C., for removing the final products. During the procedure of removing the final products from the reaction unit, the gaseous flushing medium, especially nitrogen, is applied to the reaction unit. During the procedure of removing the final products from the reaction unit, carbon can be extracted as the final product.

According to a further development of the method, extracted non-condensable gases are fed to the heating system for combustion within the heating system and thus for heating the reaction unit, and/or to a combined heat and power station for generating thermal energy and electric energy.

The method is preferably operated with at least four reaction units simultaneously and in a modular manner with the following steps:
charging a first reaction unit, while a second reaction unit, which is already charged, is preheated,
feeding a third, charged and preheated reaction unit to the heating system and heating the reaction unit in order to carry out the charring and distillation process; and
cooling and emptying a fourth reaction unit in which the charring and distillation process is completed.

The reaction unit can be charged with raw materials of a mass in the range from 2.5 to 3 t and advantageously remains in the heating system for a period of about 2.5 to 3.5 h. The reaction temperature within the reaction unit is preferably between 350° C. and 800° C., in particular 550° C.

The energy consumption for a process run with a reaction unit equipped in particular with used tyres amounts to 60 kWh to 80 kWh. A number of twelve reaction units and nine passes per day results in a daily energy demand of 6,480 kWh to 8,640 kWh. With an average of 223 production days per year, the annual energy demand is therefore 1.445 MWh to 1.927 MWh. By contrast, the values of the generated energy for electricity and heat are each about 10.5 MWh per year.

The method is based on a charring/distillation process, so that the device for the material treatment of raw materials is an industrial charring/distillation module, also referred to as a VDI module.

In order to carry out the method effectively, the device was based on the design with modules in order to thus optimise or maximise the throughput and to be able to adapt it to the current demand.

Further advantages of the device and the method compared to the prior art can be summarised as follows:
no pre-sorting of raw materials,
treatment of raw materials, in particular of
   waste rubber products, such as used tyres, rubberised chain links, steel rope-reinforced rubber belts and conveyor belts, wherein the products, in order to obtain their structure, are treatable in their substantially initial form, i.e., for example, neither crushed nor shredded, and thus not crushed, or compacted,
   organic and renewable raw materials, such as wood in all forms, in particular beech and oak, bamboo and peel and fruit, such as coconut peel and orange peel,
   animal waste, such as bones and carcasses,
   contaminated carbon,
   contaminated soils or other materials, for example after oil spills,
   substantially uncrushed or undismantled and thus complete end-of-life vehicles; and
   carbon composite materials, in particular with carbon fibres, especially from the automotive industry,
ecological, economic and carbon dioxide-free and thus sustainable technology with very low energy consumption.

The various method parameters, such as the temperatures and the duration of the process and the flushing with gaseous flushing medium, and associated therewith the performances of individual components, such as the heating system, the conveying devices of the device for forced cooling of the distillation unit, such as the fans or the at least one pump, and the extraction device, are dependent on the raw materials to be treated within the reaction unit. Thus, the methods or devices with the corresponding control programmes stored in the control device can be distinguished as follows:
a) device and method for the material treatment of tyres,
b) device and method for the material treatment of rubberized chain links,
c) device and method for the material treatment of conveyor belts,
d) device and method for the material treatment of complete vehicles or crushed vehicles of the automotive industry,
e) device and method for the material treatment of renewable raw materials such as wood and bamboo, and biowaste such as coconut shells and orange shells,
f) device and method for the material treatment of animal waste,
g) device and method for the material treatment of bitumen or asphalt,
h) device and method for the material treatment of energy storages, in particular batteries, especially from the automotive industry,
i) device and method for the material treatment of electronic components such as computers, mobile phones, laptops and smartphones, and
j) device and method for the material treatment of contaminated carbon and soils contaminated with pollutants for reactivating the carbon.

Depending on the raw materials to be treated, the raw materials are advantageously mixed in certain ratios with respect to one another within the reaction unit, for example tyres and batteries, in order to influence method parameters or final products.

The table below lists the recovered raw materials in mg/kg. The third and fourth columns list the raw materials according to a device and a method according to h) and the fifth column lists the raw materials according to a device and a method according to i), while the sixth and seventh columns list the raw materials according to a device and a method according to d) and the eighth column lists the raw materials according to a device and a method according to a).

| | Raw | | | | | | |
|---|---|---|---|---|---|---|---|
| | material/ | E-block | E-block | E-scrap | Smart | Smart | waste |
| | mg/kg | 36 kg^{1, 2, 3} | 500 kg^{1, 2, 3} | 515 kg^{1, 2, 3} | coarse^{1.2.3} | fine^{1.2.3} | tyres^{1.2.3} |
| Ag | silver | <1 | <5 | 600 | <1 | <1 | <1 |
| Al | aluminium | 106.000 | 120.000 | 73.000 | 8.300 | 7.500 | 2.500 |
| As | arsenic | <1 | <5 | 140 | <2 | <2 | <1 |
| Ba | barium | 19 | 9 | 2.000 | 54.000 | 40.000 | 18 |
| Be | beryllium | <1 | 10 | 91 | <1 | <1 | — |
| Bi | bismuth | <4 | <16 | 510 | 16 | 42 | <1 |
| Ca | calcium | 1.400 | 1.600 | 11.000 | 45.000 | 40.000 | 6.900 |
| Cd | cadmium | <1 | <2 | 51 | <1 | <1 | 1 |
| Co | cobalt | 54.000 | 60.000 | 36.000 | 83 | 110 | 108 |
| Cr | chromium | 74 | 69 | 15.000 | 190 | 230 | <1 |
| Cu | copper | 103.000 | 110.000 | 102.000 | 560 | 570 | 33 |
| Fe | iron | 28.000 | 17.000 | 122.000 | 6.500 | 6.300 | 364 |
| Ga | gallium | <21 | <28 | 240 | <2 | <2 | <1 |
| Ge | germanium | <6 | 260 | 350 | <1 | <1 | <1 |
| Hf | hafnium | <2 | <9 | 76 | <1 | <1 | — |
| In | indium | 90 | <106 | 150 | <5 | <5 | — |
| K | potassium | <38 | <86 | 350 | 1.300 | 1.200 | 1.210 |
| Li | lithium | 30.000 | 33.000 | 7.800 | 7 | 10 | 10 |
| Mg | magnesium | 710 | 990 | 5.600 | 35.000 | 34.000 | 850 |
| Mn | manganese | 46.000 | 50.000 | 12.000 | 140 | 110 | 2 |
| Mo | molybdenum | <1 | <4 | 190 | 7 | 3 | 4 |
| Na | sodium | 180 | <14 | 1.900 | 1.500 | 2.600 | 1.300 |
| Nb | niobium | <3 | <11 | 230 | <2 | <2 | — |
| Ni | nickel | 137.000 | 150.000 | 14.000 | 190 | 180 | <1 |
| P | phosphorus | 5.300 | 6.900 | 2.800 | 500 | 800 | 364 |
| Pb | lead | 16 | <10 | 310 | 62 | 150 | 28 |
| Re | rhenium | <1 | <6 | 130 | <1 | <1 | — |
| S | sulphur | — | — | 2.600 | 24.000 | 22.000 | 25.500 |
| Si | silicon | — | — | 27.000 | 67.000 | 63.000 | 43.500 |
| Sb | antimony | <2 | <9 | 150 | 170 | 160 | — |
| Se | selenium | <9 | <46 | 89 | <1 | <1 | — |
| Sn | tin | 110 | 160 | 5.300 | 110 | 120 | 2 |
| Sr | strontium | 8 | 31 | 2.500 | 790 | 650 | 6 |
| Ta | tantalum | <5 | <9 | 1.400 | <3 | <3 | — |
| Ti | titanium | 240 | 280 | 1.500 | 3.200 | 3.100 | 208 |
| Tl | thallium | — | <15 | 180 | <14 | <14 | <1 |
| V | vanadium | <5 | <3 | 190 | 14 | 15 | 6 |
| Zn | zinc | 250 | 300 | 4.700 | 14.000 | 17.000 | 39.500 |
| Zr | zirconium | 36 | 55 | 680 | 16 | 12 | — |
| C | carbon | 29.1% | 29.5% | 10.2% | 49.7% | 49.3% | |

¹Ground to particle-size <0.1 mm - results head space GC-MS screening and thermogravimetry results
²TGA graph, results GC-MS screening

Trace elements using ICP OES according to HN03/HF acid digestion—SOP 671 (679)

In the method according to h), the raw materials of which are listed in the fourth column of the table, battery blocks, also referred to as energy blocks, from the automotive industry with a mass of 500 kg and used tyres with a mass of about 500 kg were used as starting material. Prior to the process, the steel sheaths including screws with a mass of about 60 kg were removed from the battery blocks and the remaining 440 kg of starting material were layered onto a separate screen in order to avoid mixing the battery blocks with the used tyres within the reaction unit. The residues of the processed battery blocks removed from the reaction unit after the end of the completed process had a mass of 220.9 kg and were shredded to a uniform size in the range from 0.2 mm to 0.5 mm for further analysis. From the analytical data presented in the table, it appears that all inorganic or metallic components of the battery blocks are detected at a recovery rate above 98.5%. The metals and inorganic components, such as cobalt, nickel, magnesium, copper, niobium and lithium, can be recovered by proven metal refining.

In the case of the method according to (i), the raw materials of which are listed in the fifth column of the table, the starting material used was electronic waste, such as televisions, drills and cables, with a mass of 500 kg, electronic scrap, such as computers in the form of laptops and mobile phones, with a mass of 15 kg, and used tyres with a mass of approximately 500 kg. The computers and mobile phones were placed separately in a metal box in the reaction unit in order to avoid mixing with the other starting materials. The residues of the processed computers and mobile phones removed from the metal box after the end of the process had a solid mass of 7.7 kg and were shredded to a uniform size in the range of 0.1 mm for further analysis. Optical emission analysis revealed a high recovery rate of the metals, such as cobalt, chromium, lithium, nickel, cadmium, tantalum, gallium, germanium, manganese, rhenium, strontium and zirconium, which are to be recovered by proven metal refining. A recovery rate or recycling rate of 98% is observed.

In the method according to (d), the raw materials of which are listed in the sixth column of the table, a complete Smart vehicle with a mass of 750 kg was used as the starting material for the process. Before the process, only the liquids, such as the cooling fluid and the brake fluid, the engine oil and the petrol, as well as the battery were removed. The solid residues of the processed complete vehicle removed from the reaction unit after the end of the completed process had a mass of 450 kg. The mass was composed of 30% carbon and 70% metals, such as steel, spring steel and precious metals. In addition, about 250 kg to 270 kg of light oil were recovered. The proportion of residual gas was about 6% to 8%. This results in a recovery rate or utilisation rate of 95%.

In the case of samples of rapeseed, unground or ground, the method according to DIN/EN 12879 determines carbon contents between 98.8% and 99.8%, while with the same method, in the case of samples of rapeseed pellets for carbon black, carbon contents in the range from 79.7% to 81.0% are determined, in the case of samples of plastic bottles, a carbon content of 99.1% is determined, in the case of samples of oak wood, a carbon content of 98.5% is determined, in the case of samples of industrial waste, a carbon content of 99.4% is determined and in the case of rubber waste, a carbon content of 99.4% is determined. For samples of rapeseed pellets for oil, a carbon content of 99.5% is determined.

The carbon/hydrogen and nitrogen content according to ASTM D5291 and the oxygen content according to a method based on ASTM D5622 are in each case determined with VARIO EL Cube from Elementar, the fluorine content and the chlorine content are determined by means of pyrolysis ion chromatography with the Analytik Jena combustion module, absorption module 920 or ion chromatograph 930 Compact IC Flex.

Vaporisable fractions up to 200° C. are determined by means of Head space GC-MS screening with Trace GC Ultra in connection with Thermo Scientific's DSQ II mass spectrometer.

Hydrofluoric acid and nitric acid are determined by microwave digestion for ICP OES with Ofen Model StarT from MWS Gmbh, while trace elements, in particular inorganic fractions, are determined by means of ICP OES with ICP OES Arcos from Spectro.

Thermogravimetric analyses are performed with Hi-Res TGA 2950 from TA Instruments.

Further significant advantages are that the steel-rubber composites which have hitherto only been separable with high energy expenditure can be separated without significant use of external energy. The resulting products can be returned to high-quality use in the spirit of an efficient circular economy, which helps to conserve resources. In addition, novel uses of the materials obtained by the method are possible, wherein the resulting products are based on different percentage distributions, which in turn are based on the raw materials employed in different ways. The resulting products include:
light oil, for example with a density of approximately 927 kg/m³ at 15° C., a viscosity of 4.74 mm^{2/}s and a flash point below 21° C.,
gas,
metals, mainly steel or iron and titanium, and
amorphous, inorganic carbon or carbon agglomerates.

The amorphous, inorganic carbon produced by the method for the material treatment of carbon-containing raw materials has, according to the design, a structure of a three-dimensional arrangement of carbon nanoparticles as agglomerates and, depending on the starting raw materials, advantageously a degree of purity in the range from 95% to 99.9%. The carbon nanoparticles are cross-linked without long-range order, do not exhibit any large-scale graphitic arrangement and are not arranged as nanotubes.

The carbon formed with the structure of three-dimensionally arranged nanoparticles is produced industrially by means of the device or the method for the material treatment of raw materials and thus has a significant economic advantage over carbons obtained or produced in the laboratory which are known from the prior art. The purity of the carbon is significantly influenced by the flushing with gaseous flushing medium, in particular during the charring and distillation process or the cooling of the reaction unit.

Depending on the starting materials, the carbon produced in the method for the material treatment of raw materials according to the invention has a BET surface area determined by the method according to DIN ISO 9277 of greater than 2,500 m²/g BET, in particular up to 9,500 m²/g BET, specifically greater than 3,500 m²/g BET or greater than 4,000 m²/g BET, in particular in the range from 4,200 m²/g BET to 4,500 m²/g BET and thus a very high adsorption capacity without release of substances to the environment. The environment is thus not polluted, for example by leaching.

The carbon produced by the method preferably has a density of about 66 kg/m³ and can advantageously be formed with a greater tensile strength than alloyed steel.

The carbon obtained in this way can have an electric conductivity in the range from 4.5·10⁷ Ωm to 5.8·10⁷ Ωm. The electric conductivity is determined by the method according to DIN EN ISO 15091.

The carbon produced in the method according to the invention for operating the device for the material treatment of raw materials is not soluble in concentrated or dilute cold acids, such as sulphuric acid, nitric acid, hydrochloric acid, and is not attacked by alkali solutions. Nitric acid is spontaneously decomposed into water and nitrous gases, which may indicate a catalytic effect. Neither polar organic solvents nor nonpolar solvents are capable of dissolving the carbon.

The amorphous carbon produced by the method can be used, for example, in the food industry and in medicine, in decalcification systems, for the production of diamonds, as a filler for rubber in rubber production and tyre production, in aircraft construction, in the construction industry and for the production of storage systems for electric energy, such as accumulators or batteries or capacitors.

Since the amorphous carbon produced and optionally purified by the method has no acute, direct cell toxicity, the use of the carbon in medicine is possible. Incubation of cardiomyocytes with the carbon results in that the cardiomyocytes are not negatively influenced and their contractility is maintained.

The carbon can be employed for hemoperfusion/adsorption and thus for purifying blood, also referred to as dialysis. This involves the removal or reduction of harmful plasma components, which are produced either as a result of pathological changes, due to excessive absorption into the organism or due to lack of elimination in the event of a failure of kidney function or of liver function.

The blood to be purified is passed through a cartridge filled with the carbon. The cartridge has a form which allows the largest possible surface for the contact of the carbon with the blood. The outlet of the cartridge is provided with a doubly secured filter membrane so that no carbon particles can pass through the outlet and the carbon remains securely in the cartridge. The filter membrane is configured in such a way that it allows the cellular components of the blood, such as red blood cells with a size of about 7.5 μm, blood platelets with a size in the range of 1 μm to 4 μm, white blood cells with a size of 7 μm to 20 μm, or essential plasma components, such as albumin 40,000 dalton to 50,000 dalton, to pass. According to an alternative embodiment, the carbon is securely fixed to a surface by a binding method. Elements coated with the carbon are arranged inside the cartridge through which blood flows.

The carbon can also be used for topical application to the skin, in particular for wound healing, specifically for treating wounds and wound surfaces. The carbon can be fixed on synthetic surfaces, such as plaster materials.

In addition, the carbon is suitable for oral absorption as an antidote, as a carrier molecule, for example for antibiotics, for coating for improved lubricity and for implant coatings.

The carbon can also serve as an application in poisonings, in particular for the primary and secondary removal of poisons in poisonings with acids or alkalis, cyanides, alcohols, such as ethanol, methanol, and glycols, organic solvents, such as acetone and dimethyl sulfoxide, inorganic salts or metals, such as lithium, iron or other heavy metals, such as lead or mercury. The carbon can be administered in the form of tablets or powders or granules. In this case, the carbon can be taken up in a slurry in a liquid. The adsorption capacity of the carbon for fats and other substances could also lead, for example, to unburdening the liver from part of the detoxification function and the pancreas from part of the secretory function.

As mentioned above, the amorphous carbon produced by the method can also be used as a storage element, in particular as a component of an electric energy store, such as a battery or a capacitor, or as a component of a data store. The storage element can then be used, for example, in a motor vehicle, in the avionics or the satellite electronics.

Thus, so-called supercapacitors, or in short supercaps, represent an electric storage device or an electric energy store as a connection of a capacitor and a chemical battery as electrochemical capacitors. As is known, an electromagnetic energy is stored in a capacitor between two surfaces. With the large BET surface as a mass-related specific surface of the carbon with the structure of the three-dimensional arrangement of carbon nanoparticles, a high energy density is made possible in a minimum space.

With the chemical extension to the supercapacitor, the energy capacity is increased on the one hand and a higher stability is achieved on the other hand. The stability refers to an automatic self-discharge of the energy store, which is prevented in the case of a high stability.

According to the invention, a storage device is formed as an electric energy storage device in the form of a double-layer capacitor with a symmetrical structure with a housing from the outside to the inside and a collector with an electrode formed as a carbon layer and a separator with an electrolyte. The carbon layer is formed from a carbon produced by the method for the material treatment of raw materials with the structure of the three-dimensional arrangement of carbon nanoparticles.

The carbon can be employed as a filter for water treatment or for gas purification in exhaust air plants. With the help of the filters, salt water can advantageously be converted into fresh water and oil, petrol or acid or iodine can be filtered out of water. For example, the carbon is mixed with cellulose for the desalination of salt water—the filter of this type then has the form of a bag, especially a filter bag.

A study of a mixture of two litres of tap water and 500 ml of betadine, i.e. iodine mixed with 500 mg of carbon, shows a value of less than 0.1 mg/litre of I₂ (iodine). The same result is achieved with subsequent examinations of the mixture, which are staggered by three months in each case. The investigations based on a photometric analysis of iodine show that the carbon introduced into the mixture binds the iodine and does not release it again there.

The introduction of carbon into water also improves water quality with regard to oxygen content—it promotes oxygen exchange, for example when used in an aquarium. A further advantage is that, for example, coli bacteria become active only at elevated temperatures of the water of about 36° C. to 38° C. and above. At temperatures below the indicated range, on the other hand, no coli bacteria are formed.

For example, a water filter system has a filter element consisting of 40 kg of the carbon, which is divided into four units. The water filter system is used to clean a volume of about 4 million litres of water, for example a pool of water in a bath or a lake.

The amorphous carbon produced by the method can also be used as a filter element for purifying air in air-conditioning systems, for example of real estate, such as hospitals, residential buildings, factories, halls or the like, and of mobile units, such as vehicles or aircrafts. In addition, the carbon can be used as an air filter element in respirator masks or an exhaust device, for example of a motor vehicle.

Due to its properties, the carbon is suitable for oil control. The carbon floats on the surface of the water and traps oil on the water, as can occur in maritime accidents, for example. Water contamination can be combated or prevented. Filter material of 1 kg of carbon absorbs about 3.33 litres of oil. Consequently, if 1 litre of oil contaminates about 1 million litres of water, 10 litres of oil can be absorbed with a filter material of 3 kg of carbon and 10 million litres of water can be purified in this way.

However, the carbon can also be used to clean up soils contaminated with mineral oil, i.e. in soil contaminations, or in other cases of oil damage or contaminated substances.

The carbon has a very good elution behaviour and consequently prevents the dissolution of already adsorbed substances, in particular in water. This avoids a pollution of the soil and groundwater due to possible leaching of pollutants from the carbon.

The carbon is also advantageously used for fire-fighting on land and in water, in particular for fighting burning oil. The carbon can thus be used as an extinguishing agent on the one hand, wherein oxygen is removed from the fire by covering it with a corresponding amount of carbon, so that the flame suffocates. On the other hand, the oil is bound simultaneously with the carbon. The amorphous carbon produced by the method can also be employed to extinguish forest fires, hinders the spread of harmful fungi from the wood ash and filters released toxins, such as lead, mercury, sulphur and dioxin.

A further application of the carbon serves for fire protection and thermal insulation up to at least 3,500° C. Thermal insulation is also understood to be insulation at very low temperatures, i.e. cold insulation. Studies with plasma jets as thermal excitation on the one hand and with liquid nitrogen on the other hand show that carbon-coated base materials not only withstand temperatures in the range from 2,000° C. to 12,000° C. on the one hand and up to −196° C. on the other hand, but also represent thermal insulation.

A coating of a wide variety of materials, for example of glass, wood, metals, plastics or other building materials, such as gypsum, clay, concrete or the like, or also paper or cardboard, with the carbon leads, for example, to an increase in the fire resistance associated with a thermal insulation effect. The layer of carbon can have a thickness in the range from 2 μm to 10 μm, in particular in the range from 2 μm to 6 μm.

The carbon can also be mixed with materials. In this case, for example, a mixture of cement and carbon in a volume ratio in the range from 2:1 to 5:1 has very good properties with respect to the thermal resistance or the thermal insulation. For example, a lightweight sheet of carbon cement with a mixture ratio of carbon and cement of 3:1, a thickness of 10 mm up to temperatures of about 2,000° C., is heat resistant. In this case, the plate is heated on a first side with a propane gas flame to a temperature between 1,500° C. and 2,500° C., without there being any noticeable heat development on the second side opposite the first side, which is to be understood as thermal insulation.

Depending on the carrier material, a mixture of the material with the carbon can be heat-resistant at a proportion of 20% carbon up to a temperature of at least about 2,500° C.

In addition to cement or concrete, for example, gypsum and clay, paints and varnishes and sawdust can also serve as a mixture component with the carbon.

In addition, the radiation-repellent carbon can be employed in installations and devices with the necessary radiation protection or for radiation shielding. The advantageous properties, such as the radiation resistance and the fire resistance, lead, for example, to employment in the construction of enclosures for nuclear reactors or to the prevention of the penetration or passing of X-rays.

A further area of application for the carbon as a very good water reservoir and nutrient reservoir is the provision of water-holding layers. This leads to a water saving of 60% to 80% in cultivated areas, for example for the production of foodstuffs, in particular in internal irrigation.

By employing carbon, for example below sandy layers, water and plant nutrients can be stored and poor, inferior soils can be used as a location for vegetables and other agricultural products. This application is therefore of great advantage for the reclamation of desert areas, in horticulture and in agriculture. It is also known that the carbon does not release any substances to the water, so that no pollution of the soil and groundwater occurs due to a leaching of pollutants.

The amorphous carbon produced by the method can consequently be used in the field of crop production, agriculture and environmental maintenance of landscape construction in order to promote the water-holding capacity of the soil by enriching the uppermost soil layers with the carbon.

By improving water storage, the optimal supply of water to the plants and thus earliness of and an increase in the quality and quantity of the yield is brought about. Since the water seepage into deeper layers is prevented and kept in the region of the roots of the plants, longer-lasting dry periods can be bridged at the same time. The described effect, which also serves to green deserts, steppes and savannas, can be achieved in particular by introducing one or more layers of carbon over a large area into light soils at a depth of about 20 cm to 30 cm.

The carbon layer influencing the pH value improves the aeration of the soil by releasing bound oxygen and nitrogen, promotes the accumulation of microorganisms and optimises the living conditions of the microorganisms. In addition, the carbon layer improves the supply of minerals, trace elements and micronutrients to soils and plants and promotes the acceleration of the ripening process and the taste of the fruit without toxic impairments. The carbon layer supports the regulation of the temperature conditions in the soil and improves the buffering properties of the soil.

In addition, the carbon can be used as a sustainable natural straw stabiliser in grain production. The carbon takes over the function of legumes without having to grow them in real terms in the crop rotation.

The recovered light oil is used, for example, in the chemical industry, in particular as a raw material for base chemicals, and in the pharmaceutical industry, for generating thermal energy and electric energy, for example by means of a CHP, while the gas can be used for generating thermal energy and electric energy, for example by means of a gas turbine and generator, or for recycling and use in the process. The recovered metals, such as steel, can be recycled to the steel industry, where the physical and chemical properties of metals can be maintained by very low process temperatures.

Further details, features, and advantages of the invention will become apparent from the following description of exemplary embodiments with reference to the accompanying drawings. In the drawings:

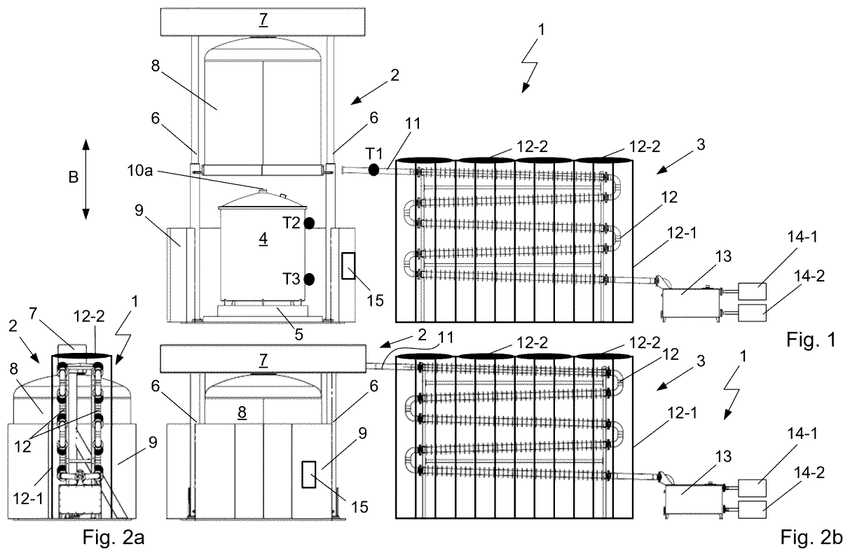

FIG. 1: industrial charring/distillation module as a device for the material treatment of raw materials in the open state in front view,

FIG. 2a: industrial charring/distillation module as a device for the material treatment of raw materials in the closed state in side view, and

FIG. 2b: in front view,

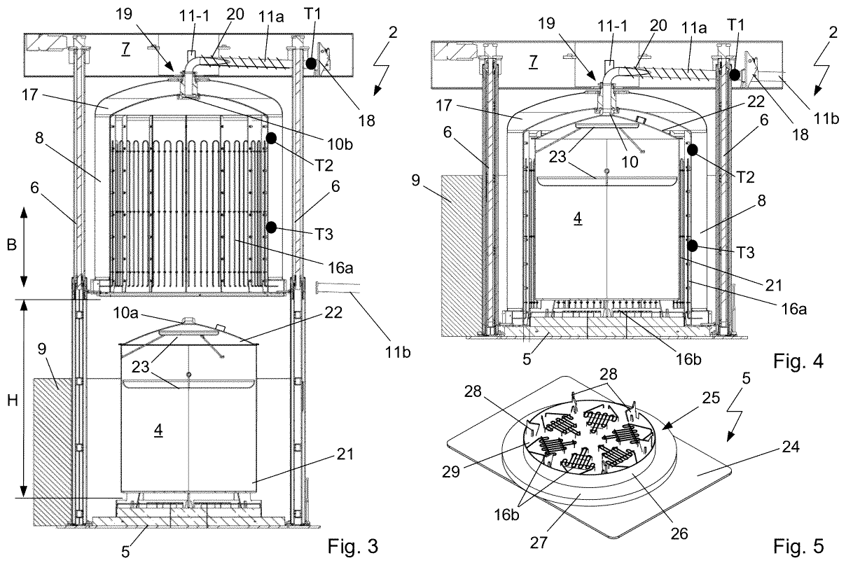

FIG. 3: sectional representation of the heating system in the opened condition,

FIG. 4: sectional representation of the heating system in the closed condition,

FIG. 5: bottom element of the heating system,

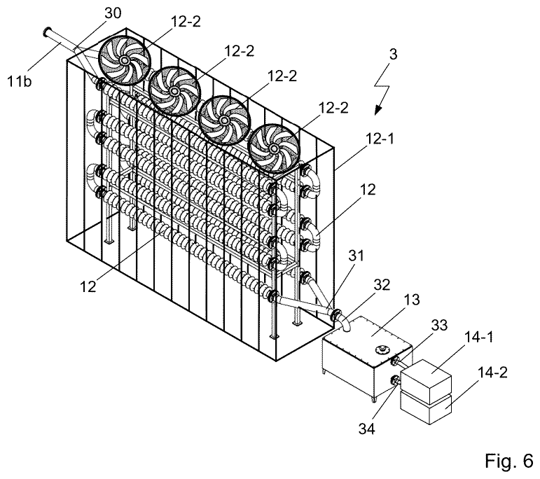

FIG. 6: distillation unit,

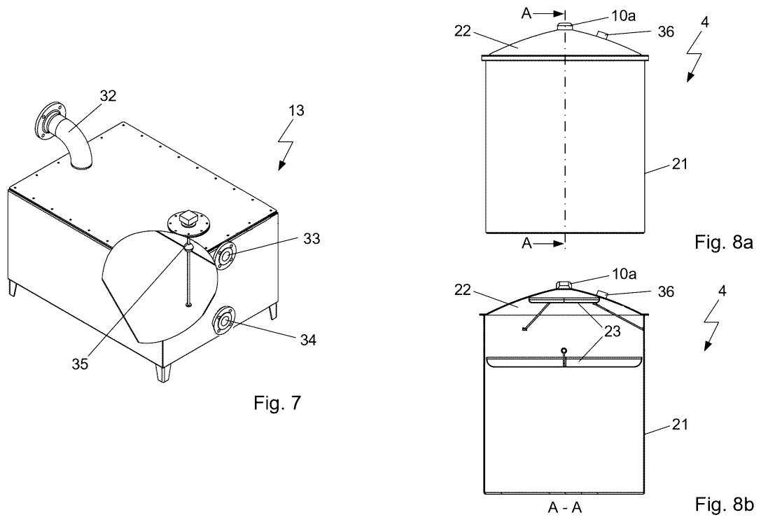

FIG. 7: oil tank, and

FIG. 8a: reaction unit in the closed state, and

FIG. 8b: sectional representation of the reaction unit in the closed state,

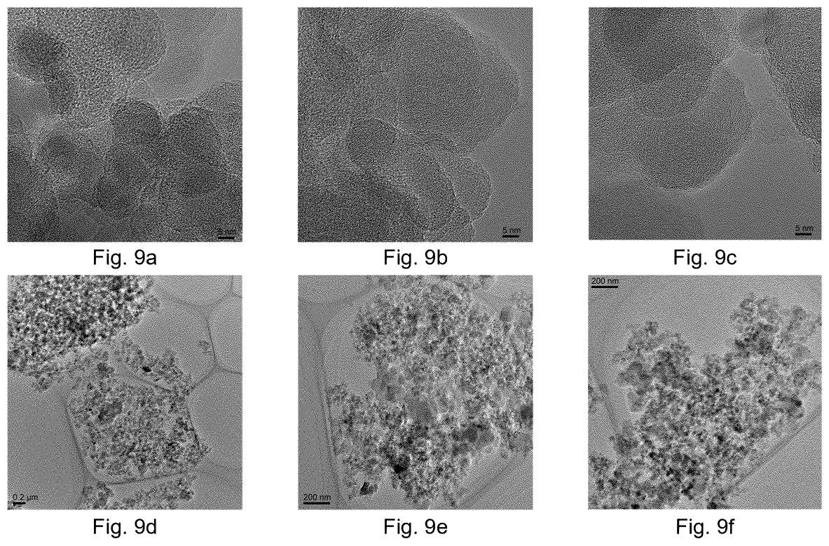

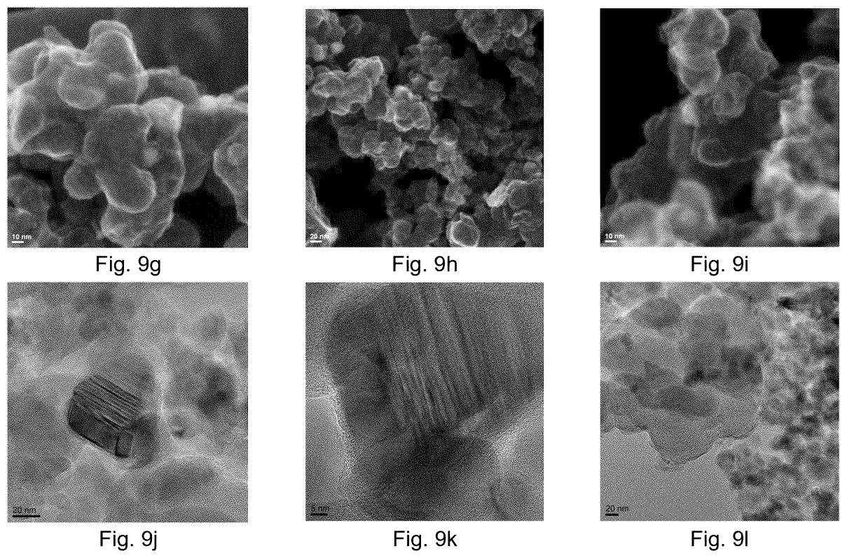

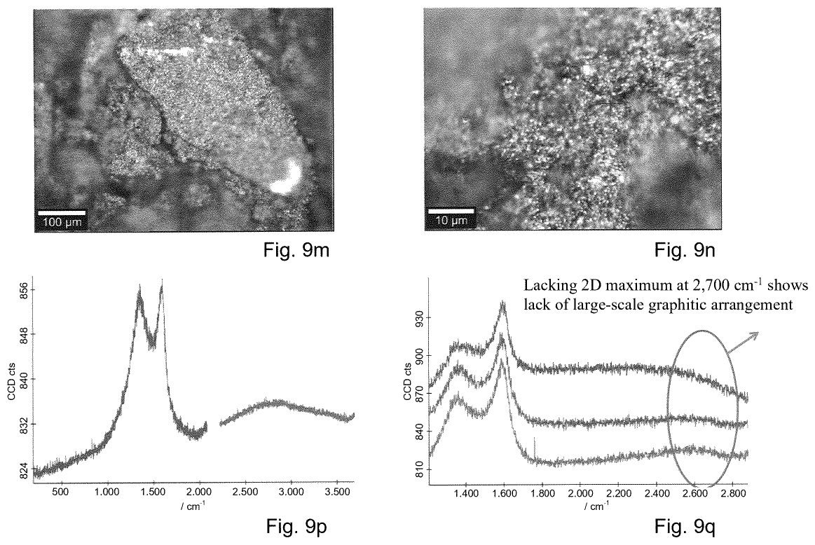

FIGS. 9a to 9n: microscopic images of carbon produced by the device for the material treatment of raw materials, and

FIGS. 9p and 9q: results of Raman spectroscopy of the carbon, and

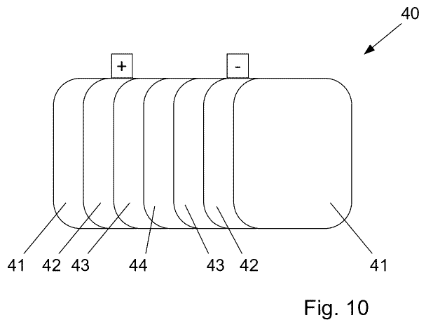

FIG. 10: an electric energy storage device as a storage device in the form of a supercapacitor in layered construction.

In FIGS. 1, 2a and 2b, an industrial charring/distillation module is represented as a device 1 for the material treatment of raw materials. FIG. 1 shows the device 1 in the open state in front view, while FIG. 2b shows the device 1 in the closed state in front view and FIG. 2a in side view.

The device 1 has a heating system 2 and a distillation unit 3. The reaction unit 4 charged with raw materials is preheated to a certain temperature in a non-represented preheating device and subsequently heated further in the heating system 2. The reaction unit 4 can be charged with a mixture of different raw materials, so that no pre-sorting of the products is necessary. After preheating, the reaction unit 4 is brought into the opened heating system 2 and positioned on the bottom element 5 of the heating system 2.

The head element 7 and the jacket element 8 of the heating system 2, which is firmly connected to the head element 7, are held movably in the movement direction B by means of support elements 6 arranged on both sides of the heating system 2. The support elements 6 are arranged at a distance of about 2.9 m from one another. The jacket element 8 has an outer diameter of about 2.5 m.

In the first end position according to FIG. 1, the support elements 6 are extended. Thus, the device 1 has a height of about 6.70 m. The head element 7 and the jacket element 8 open the space for equipping the heating system 2 with the reaction unit 4. The heating system 2 is opened. The reaction unit 4 can be introduced into the heating system 2 or removed from the heating system 2. The movement of the reaction unit 4 can advantageously take place by means of a rail system, not represented, on which the reaction unit 4 stands. In the second end position according to FIGS. 2a, 2b, the support elements 6 are retracted. Thus, the device 1 has a height of about 3.70 m.

The jacket element 8 is seated on the bottom element 5 in such a way that the reaction unit 4 is positioned in a closed space. The heating system 2 is closed. The reaction unit 4 is surrounded at the bottom by the bottom element 5 and at the side surface and at the top by the jacket element 8.

The device 1 has temperature sensors T1, T2, T3 in the region of the heating system 2 and the distillation unit 3 for determining certain process temperatures. At least two temperature sensors T2, T3 are arranged in an intermediate space formed between the reaction unit 4 and the jacket element 8 in the closed state of the heating system 2. The temperature sensors T2, T3 are positioned, for example, from the inside of the jacket element 8 to project approximately 1 cm into the approximately 8 cm wide intermediate space. The temperature sensors T2, T3 are arranged at a vertical distance from one another in order to determine local temperature values or an average temperature within the intermediate space. The temperature within the reaction unit 4 is determined with the temperature values determined via the temperature sensors T2, T3.

The heating system 2 has an enclosure 9 in the lower region. The enclosure 9 enclosing the bottom element 5 and the side surfaces of the jacket element 8 in the closed state of the heating system 2 is opened for the purpose of equipping the heating system 2.

The gases produced in the charring process are discharged from the heating system 2 through the provided exhaust gas line 11 and are cooled down in terms of process technology. The gases are passed to the distillation unit 3 through the exhaust gas port 10a formed at the uppermost point of the reaction unit 4 and through the exhaust gas line 11 arranged in the head element 7. Subsequently, the gases flow through the cooling section 12 of the distillation unit 3. The cooling section 12 is formed from tubes according to FIGS. 1, 2a, 2b. The tubes inclined to the horizontal are provided with ribs in order to increase the heat transfer surface and thus to improve the heat transfer. The heat is transferred from the gases to the ambient air.

In order to further increase the heat output to be transferred from the gases to be cooled to the ambient air and, specifically, to improve the control of the temperature of the gases flowing through the cooling section 12 of the distillation unit 3, the cooling section 12 is surrounded by an air guide housing 12-1. Fans 12-2 are arranged on the upper side, in particular on the end face of the air guide housing 12-1 pointing upwards in the vertical direction, which fans draw in the ambient air as cooling air uniformly through the air guide housing 12-1. Alternatively, the fans can also be formed on a side surface of the air guide housing 12-1. In this case, the ambient air is directed in a targeted manner over the cooling section 12. A further temperature sensor T1 is arranged on the exhaust gas line 11 formed between the heating system 2 and the distillation unit 3 in order to determine the temperature of the exhaust gases discharged from the heating system 2.

According to an alternative embodiment, the gases within the cooling section can also be cooled with a heat transfer fluid other than air, for example water. In this case, instead of tubes, the cooling section is formed with ribs of coaxial tubes formed on the outer jacket surface. The gases flow in the interior of the inner tube, while the preferably liquid heat transfer fluid is passed through in the intermediate space between the outside of the inner tube and the inside of the outer tube.

The cooling section 12 is formed with two tubes aligned parallel to one another. The gases are divided into two partial mass flows before entering the cooling section 12 and are mixed again after flowing through the cooling section 12.

Subsequently, the distillation products are introduced into an oil tank 13. In the oil tank 13, the oil obtained from the charring process and the subsequent distillation, which in its consistency and composition corresponds to a light oil or is very similar to the intermediates of crude oil processing, settles. The non-condensable portion of the gas is discharged from the oil tank 13. The oil tank 13, with a capacity of about 1,000 litres, also serves as an expansion vessel of the device 1.

An extraction device 14-1, in particular a pump, specifically a diaphragm pump, for extracting the gases via the surface of the oil accumulating within the oil tank 13, and an oil conveying device 14-2, in particular a pump, specifically a piston pump, for extracting the oil from the oil tank 13 are arranged on the oil tank 13. When the gases are extracted, a negative pressure is generated within the cooling section 12 of the distillation unit 3, the exhaust gas line 11 and specifically within the reaction unit 4. With the extraction device 14-1, the air and thus the oxygen as a constituent of the air are also purposefully extracted of the reaction unit 4. A vacuum can thus be generated within the reaction unit 4.

The gases extracted above the surface of the oil accumulating inside the oil tank 13 can be used directly with a combined heat and power station, referred to as a CHP, for generating thermal energy and electric energy.

The device 1 is also formed with a control device 15 for controlling the method for operating the device 1. The control device 15 determines and indicates, for example, the filling level within the oil tank 13, the flow of oil or gas and a possible defect in a line of the device 1. The control device 15 is connected to corresponding sensors. The temperature sensors T1, T2, T3 are also coupled to the control device 15. The values determined with the temperature sensors T1, T2, T3 serve to control the device 1, in particular the heating system 2 and thus to heat the reaction unit 4 and the fans 12-2 of the cooling section 12 and the extraction device 14-1. The control device 15 can be used, inter alia, to indicate the status of different heating circuits of the heating system 2 and process temperatures. The arrangement of the jacket element 8 of the heating system 2 can also be determined and represented in the open, closed and partially open states. Consequently, the control device 15 also serves to extend and retract the support elements 6 for opening and closing the heating system 2.

FIGS. 3 and 4 each show a sectional representation of the heating system 2. FIG. 3 represents the heating system 2 in the opened state and FIG. 4 in the closed state.

According to FIG. 3, the support elements 6 are fully extended. The head element 7 arranged at the upper ends of the support elements 6 and the jacket element 8 firmly connected to the head element 7 are arranged at a height H above the bottom element 5, so that the reaction unit 4 can be freely moved in the horizontal direction between the bottom element 5 and the jacket element 8.

The jacket element 8 is supported movably in the lower region against the support elements 6. By means of the lateral support against the support elements 6, a straight movement of the jacket element 8 in the movement direction B between the end positions is ensured. Canting of the jacket element 8 is avoided.

The jacket element 8 has heating elements 16a distributed uniformly on the circumference of the inner surface of the jacket. The heating elements 16a are arranged substantially in the vertical direction and are guided through the wall to the inner surface in the lower region of the jacket element 8. The heating elements 16a are each formed from two vertically aligned sections which are connected to one another at the upper end by means of a deflection.

The jacket element 8, which is open downwards in the vertical direction, is closed at the top by a hood 17 and fastened to the head element 7. The head element 7 and the jacket element 8 form a coherent unit. The hood 17 is formed at the centre point with an exhaust gas port 10b as a connection to the exhaust gas line 11a. The exhaust gas line 11a extends from the exhaust gas port 10b through the hood 17 into the head element 7. The passage of the exhaust gas line 11a through the hood 17 is sealed off from the hood 17. The exhaust gas line 11a is formed in the region of the exhaust gas port 10b with a pipe connection 19 which can vary in length in the vertical direction, for example in the form of a telescopic pipe. The tube connection 19, which is automatically adjustable in length, serves to compensate for thermal expansions of the reaction unit 4, in particular with respect to the jacket element 8 with the hood 17 of the heating system 2.

The exhaust gas line 11a is formed as a transition from the reaction unit 4 to the distillation unit 3 with a heating device 20. The electrically operated heating device 20 surrounding the exhaust gas line 11a is connected to the control device 15, as is the temperature sensor T1.

In addition, the exhaust gas line 11a has a connecting element 11-1 for connecting the exhaust gas line 11a to a device for admitting a gaseous flushing medium, for example nitrogen. The flushing medium can flow into the exhaust gas line 11a and in particular into the reaction unit 4 via the connecting element 11-1. The connecting element 11-1 is arranged between the pipe connection 19 and the region of the exhaust gas line 11a which is enclosed by the heating device 20, especially at the highest point of the exhaust gas line 11, 11a in the vertical direction.

The exhaust gas line 11a has a connecting element 18 at the distal end, starting from the exhaust gas port 10b. The connecting element 18, which is advantageously formed as a quick-connect coupling, serves to connect the exhaust gas line 11a of the heating system 2 to the exhaust gas line 11b of the distillation unit 3 in the closed state of the heating system 2 according to FIG. 4. As a result of the downward movement of the head element 7 when closing the heating system 2, the exhaust gas lines 11a, 11b on the connecting element 18 and the exhaust gas ports 10a, 10b are coupled to one another, so that a gas-tight connection is produced from the reaction unit 4 to the distillation unit 3.

The reaction unit 4 arranged on the bottom element 5 is formed with a wall 21 in the form of a hollow cylindrical vessel with an outer diameter of approximately 1.8 m, which is closed at the bottom. The open side of the wall 21 can be closed by means of a cover element 22. A seal is arranged between the wall 21 and the cover element 22, so that the reaction unit 4 is tightly closed. Screen elements 23 are formed in the interior of the reaction unit 4. The screen elements 23 are aligned in the horizontal direction and are arranged at different heights, spaced apart from one another.

In the second end position shown in FIG. 4, the support elements 6 are fully retracted. The jacket element 8 is seated on the bottom element 5 and completely encloses the reaction unit 4. The heating system 2 is closed.

The reaction unit 4 charged with raw materials is advantageously heated uniformly over the bottom and the wall 21. The heating elements 16a are used for heating via the wall 21, while heating elements 16b arranged on the bottom element 5 supply heat through the bottom to the reaction unit 4. In the closed state of the heating system 2, the heating elements 16a formed on the circumference of the jacket element 8 have equal distances from the wall 21 of the reaction unit 4. The heating elements 16a, 16b are preferably electrically operated.

The reaction unit 4 remains in the heating system 2 for a period of about 2.5 h to 3.5 h in which the main reaction and conversion of the raw materials takes place within the reaction unit 4. The reaction temperature within the reaction unit 4 is between 350° C. and 800° C., in particular between 400° C. and 600° C., specifically about 550° C., depending on the feed and depending on the final products to be produced. This temperature is determined by means of the temperature sensors T2, T3 arranged between the reaction unit 4 and the jacket element 8. This consumes an energy in the range of 40 kWh per hour. The reaction unit 4 is charged with raw materials of a mass in the range from 2.5 t to 3 t.

The gases formed during the charring process are discharged, in particular extracted, into the exhaust gas line 11 through the exhaust gas port 10 arranged on the cover element 22. In the closed state of the heating system 2, the exhaust gas port 10a of the reaction unit 4 and the exhaust gas port 10b of the hood 17 of the jacket element 8 are connected to one another in a gas-tight manner. This ensures that no gases can escape into the intermediate space between the reaction unit 4 and the jacket element 8.

Inside the reaction unit 4 there is a negative pressure with an absolute value in the range from 2 mbar to 10 mbar, specifically about 4 mbar, which is generated by the extraction device 14-1 arranged at a first outlet port of the oil tank 13 for extracting the gases via the surface of the oil accumulating within the oil tank 13. With the targeted extraction of the oxygen from the reaction unit 4, the reaction temperature or the process temperature within the reaction unit 4 is reached in a shorter time on the one hand. On the other hand, this influences the structure formation of the carbon as the final product. Another factor influencing the formation and purity of the carbon is the duration of the charring process. The longer the charring process takes place, the cleaner the carbon and can also be employed, depending on the starting materials, for medical purposes, for example. The carbon employed for medical purposes should be additionally purified, if necessary. Carbon recovered during a rather shorter charring process is preferably used, for example, as a filter material or in the construction industry.

Influencing factors on the formation and purity of the carbon also include the flushing of the reaction unit 4 with the gaseous flushing medium, in particular nitrogen, during the charring and distillation process on the one hand and during the procedure of cooling the reaction unit 4 on the other hand.

By means of the heating device 20 surrounding the exhaust gas line 11a, the exhaust gas line 11a is heated, in particular to a temperature in the range from 120° C. to 160° C., in order to reduce the temperature difference between the exhaust gas line 11a and the exhaust gas flowing through the exhaust gas line 11a. The temperature of the exhaust gas flowing through is determined by means of the temperature sensor T1. The heating device 20 serves to prevent premature condensation of the exhaust gas prior to entry into the distillation unit 3 and thus also an undesired clogging of the exhaust gas line 11a. The heating of the exhaust gas line 11a supports the outflow of the exhaust gas from the reaction unit 4.

In FIG. 5, the bottom element 5 of the heating system 2 is represented. The bottom element 5 has a bottom plate 24 and a centring device 25 for the jacket element 8, heating elements 16b and support elements 28 for holding the reaction unit 4. The bottom element 5 is substantially formed from ceramic in order to ensure thermal insulation towards the outside, in particular towards the bottom. In combination with the thermal insulation of the jacket element 8, the heat loss of the heating system 2 is thus minimised.

The reaction unit 4 stands on the support elements 28 of the bottom plate 24. The support elements 28 are formed and arranged in such a way that the reaction unit 4 is aligned centrally with the bottom element 5 when it rests on the support elements 28.

The centring device 25 is formed in the form of a circular disk with a shoulder. Consequently, the disk has two regions with different diameters. The circular surface arranged between the regions serves as a sealing surface 27.

The outer circumference of the region of the disk with the smaller diameter is smaller than the inner circumference of the wall 21 of the reaction unit 4 or of the jacket element 8. In the closed state of the heating system 2, a gap is formed between a jacket surface 26 of the region of the disk with the smaller diameter and the inner side of the wall 21. The jacket element 8 stands on the sealing surface 27 of the bottom plate 24, so that the space enclosed by the jacket element 8 and the bottom plate 24 is tightly closed. Seals are arranged on the corresponding surfaces of the bottom plate 24 and of the jacket element 8 in order to seal the enclosed space. In addition, the jacket element 8 is pressed and held onto the sealing surface 27 of the bottom plate 24 with a pressure in the range from 1 bar to 2 bar.

Since the support elements 6 are also fastened to the bottom plate 24, the bottom plate 24 carries the entire heating system 2.

The heating elements 16b are arranged substantially in the horizontal direction, on a terminal surface 29 of the centring device 25 and guided vertically through the terminal surface 29. The heating elements 16b, which are curved in a meandering manner, each have the form of a hand with five fingers. The length of the fingers increases from the outside to the inside, so that the middle finger has the greatest length. The heating elements 16b are aligned symmetrically to one another, with the tips of the fingers pointing towards the centre point of the terminal surface 29.

The support elements 28, on which the reaction unit 4 stands, project in the vertical direction beyond the heating elements 16b, so that the bottom of the reaction unit 4, which stands on the support elements 28, is arranged above the heating elements 16b. The heating elements 16b each have the same distance from the bottom of the reaction unit 4, in order to ensure a uniform introduction of heat through the bottom of the reaction unit 4.

The centring device 25, the support elements 28 and the heating elements 16b are arranged concentrically around the centre point of the bottom plate 24.

FIG. 6 shows the distillation unit 3, having the exhaust gas line 11b, the cooling section 12 with the air guide housing 12-1 and the fans 12-2, and the oil tank 13 with the extraction device 14-1 and the oil conveying device 14-2 in the order of the flow direction of the final products.

The gases discharged from the heating system 2 are passed through the exhaust gas line 11b to the cooling sections 12, which are likewise formed from pipes. The gas mass flow is divided into two partial mass flows at a branch 30 by two tubes aligned parallel to one another. The division of the gas mass flow results in a better heat transfer from the gas mass flow to the environment in order to optimise the procedure of distillation or condensation.

In order to further improve the heat transfer, the tubes are formed with ribs in order to increase the heat transfer surfaces of the cooling sections 12. The heat output to be dissipated from the gases to be cooled, in particular the amount of condensation heat, is further increased and simultaneously controlled by the air guide housing 12-1 and the fans 12-2. The ambient air is sucked uniformly through the air guide housing 12-1 as cooling air and guided over the cooling sections 12 in a targeted manner. The corresponding output or the air volume flow of the fans 12-2 ensures that the exhaust gases flowing through the cooling section 12 of the distillation unit 3 can be liquefied at a condensation temperature in the range from 95° C. to 125° C. With the additional inflow of the cooling sections 12, the cooling sections 12 are cooled to a temperature below the condensation temperature of the gases or kept at the corresponding temperature level. With the heat output controlled in this way, a higher yield of oil is achieved with a lower yield of residual gas. The temperature is determined by means of the temperature sensor T1, according to FIG. 1, arranged on the exhaust gas line 11 formed between the heating system 2 and the distillation unit 3.

After flowing through the cooling sections 12, the partial mass flows divided up before entry into the cooling sections 12 are recombined at an opening point 31 and introduced from above into the oil tank 13 through an inlet port 32.

The oil, which has a greater density than the gas, is deposited in the oil tank 13. The non-condensable portion of the distillation products is removed in the upper region of the oil tank 13 through a first outlet port 33. For extracting the gases via the surface of the oil accumulating within the oil tank 13, the extraction device 14-1 is arranged at the first outlet port 33 of the oil tank 13. With the extraction of the gases and the thus generated negative pressure within the device 1, in particular the air and thus the oxygen as a constituent of the air is extracted from the reaction unit 4 and the charring process is influenced.

For conveying the oil from the oil tank 13, the oil conveying device 14-2 is arranged on a second outlet port 34 of the oil tank 13.

In FIG. 7, an oil tank 13 with a cut-open side surface is represented for viewing into the interior.

The inlet port 32 is arranged on the upper side of the oil tank 13 so that the distillation products flow into the oil tank 13 from above. The oil settles at the bottom of the oil tank 13, while the gases, which have lower densities in contrast to the oil, are concentrated above the oil level. The oil level in the oil tank 13 is determined and observed with a float 35. When a predetermined filling height is reached, the oil is removed from the oil tank 13 for further processing.

The gases accumulating in the upper region of the oil tank 13 are discharged through the first outlet port 33, in particular extracted by means of the extraction device 14-1, while the oil accumulating in the lower region of the oil tank 13 is extracted through the second outlet port 34, in particular by means of the oil conveying device 14-2.

In FIGS. 8a and 8b, the reaction unit 4 is represented in the closed state, wherein FIG. 8b shows a sectional view of the reaction unit 4.

The wall 21, which is in the form of a hollow cylindrical vessel and has a closed bottom, can be closed at the open side opposite the bottom by means of a cover element 22. During the procedure of closing the reaction unit 4, the cover element 22 is placed in the vertical direction on the upwardly directed end face of the wall 21. Due to its own weight, the cover element 22 is pressed against the end face of the wall 21 and bears releasably against the wall 21.

A high-temperature-resistant seal is arranged between the wall 21 and the cover element 22 for closing the reaction unit 4 in a tight manner. In the closed state, the reaction unit 4 has a height of about 2.4 m.

The cover element 22 is formed next to the exhaust gas port 10a with a connecting port 36. A device for introducing a gaseous flushing medium, in particular nitrogen, into the reaction unit 4 can be connected to the connecting port 36.

The actual charring/distillation process, in which the reaction unit 4 is arranged within the heating system 2 and is heated or is kept substantially at the desired reaction temperature, is terminated at a temperature of the exhaust gas of about 60° C. determined by the temperature sensor T1 arranged between the heating system 2 and the distillation unit 3. The reaction unit 4 is removed from the heating system 2 and has a temperature, for example, in the range from 500° C. to 600° C.

After removal from the heating system 2, the reaction unit 4 is cooled to the temperature defined as a function of the use of the product. The mixture located inside the reaction unit 4 is removed after the reaction unit 4 has been opened, i.e. after the cover element 22 has been removed. The reaction unit 4 is then fed back to the process and charged. The carbon-iron mixture is separated into its components.

The recovered unique carbon is further formed in the oxygen-free atmosphere without oxygen during the cooling procedure between 600° C. and 60° C. or 20° C. or 30° C. within the reaction unit 4. In this case, the gaseous flushing medium, in particular nitrogen, is flowed into the reaction unit 4 through the connecting port 36, which likewise influences the cooling procedure. Alternatively, the gaseous flushing medium can be introduced through the exhaust gas port 10a, to which the device for introducing the gaseous flushing medium can be connected, in particular if the connecting port 36 is not formed. The inflow of the flushing medium during the cooling procedure and thus before the emptying of the reaction unit 4 can accelerate the procedure of cooling, but serves above all for cleaning the final products and could thus also support the formation of the carbon recovered with the device 1. The flushing of the reaction unit 4 increases the purity of the final products, in particular of the carbon. Impurities are flushed out. The flushing medium flowing into the reaction unit 4 through the connecting port 36 is again discharged from the reaction unit 4 together with the impurities through the exhaust gas port 10a formed in the cover element 22. The reaction unit 4 is opened at a temperature inside the reaction unit 4 in the range from 20° C. to 60° C., in particular in the range from 30° C. to 60° C.

During the procedure of opening the reaction unit 4, the cover element 22 is raised in the vertical direction and removed from the reaction unit 4 in such a way that the reaction unit 4 can be emptied and subsequently charged again. The reaction unit 4 can also be charged with the flushing medium during the procedure of emptying in order to achieve a desired purity of the final products, in particular of the carbon. The carbon is preferably extracted during emptying of the reaction unit 4.

The charring/distillation process for the material treatment of the raw materials simultaneously involves four reaction units 4 made of high-temperature-resistant steel, each with a filling quantity in the range from 2.5 t to 3.5 t (75% mechanically, 25% automated). While the first reaction unit 4 is charged, the second reaction unit 4, which is already charged, is preheated. Meanwhile, the third reaction unit 4 is already fed to the heating system 2 and is heated so that the actual charring/distillation process takes place. Meanwhile, the fourth reaction unit 4 is cooled and subsequently emptied.

By using the modular system, for example with four reaction units 4, the throughput can be increased stepwise and flexibly adapted to the respective demand. The entire process takes place quasi-continuously.

In FIGS. 9a to 9n, microscopic images of carbon produced with the device 1 for the material treatment of raw materials are shown. A structure of the carbon can be seen from the images produced using a transmission electron microscope, referred to as TEM for short. Transmission electron microscopy is used to detect and characterise the structure and particle size of substances and substance mixtures in the nanometre range.

The images show a very finely divided, three-dimensional, homogeneous and pseudo-crystalline structure of the primary carbon particles in the subnanometre range with a very large inner surface. The carbon particles are partially recognisable as larger agglomerates with the same surface structure.

FIGS. 9p and 9q show results of a Raman spectroscopy of the carbon. The missing 2D maximum at 2,700 cm-1 shows the absence of a large-scale graphitic arrangement. The carbon produced with the device 1 for the material treatment of raw materials is amorphous, inorganic carbon in which the nanoparticles are cross-linked without long-range order. The carbon neither has nanotubes nor is structurally similar to graphene.

The images of a Raman spectrum as well as the determination of the intensity and width of G-Raman and D-Raman bands can be done with Confocal RAMAN Microscope in Via by Renishaw with 532 nm and 785 nm lasers.

In FIG. 10, an electric energy store is shown as a storage device 40 in the form of a supercapacitor in layered form. The supercapacitor can have a symmetrical construction as a storage device 40 with a double layer of carbon. The structure of the negative pole and the positive pole are identical. The poles can be defined during the first loading process.

The storage device 40 has a housing 41 and a collector 42 with an electrode 43 in the form of a carbon layer from the outside to the inside. A separator 44 and an electrolyte are provided in the interior. From the inside to the outside, the electrode 43, a collector 42 and the housing 41 are consequently arranged on both sides of the separator 44 with the electrolyte. The storage device 40 consequently has a double layer.

The housing 41 can be formed as a laminating film of polyester, referred to in short as PET, which has an adhesive layer of ethyl vinyl acetate, referred to in short as EVA, on an inwardly directed side. The collectors 42 with the electrodes 43 are pressed together with the housing 41. The interior of the storage device 40 is kept free of oxygen in order to prevent oxidation.

The ions are deposited on the collectors 42 each preferably formed from a brass foil having a thickness of approximately 0.2 mm and a surface of 10 cm². The collectors 42 establish the electric connection for the electric current from the connections of the storage device 40 to the electrodes 43. The collectors 42, in particular the electrodes 43, are compatible with the electrolyte so that an undesired reaction is avoided.

The crushed carbon, in particular previously processed with a mixer and subsequently further refined with a mortar, is mixed with methyl alcohol, specifically with 99% methyl alcohol, and applied to the ground-on and subsequently degreased brass film. The mixture of carbon and methyl alcohol can be applied with a spraying tool, in particular an air spray gun, also referred to as an “airbrush gun”. Since the methyl alcohol evaporates completely after application to the brass foil, the mixing ratio of carbon and methyl alcohol is irrelevant and can be adapted to the spraying tool. The injection mould can be used to apply several layers, preferably a first and a second layer.

For a further layer, soda water glass, in particular 10% soda water glass, is mixed with demineralised water in a ratio of 1:1. Carbon is then added to the mixture of soda water glass and demineralised water. 10 ml of carbon, which is determined as a flaky volume, are added to 40 ml of solution. The finished mixture is likewise applied by means of the injection tool, preferably in each case in two layers, to the brass foil, in particular the already present layers of carbon. This binds the lower layers which absorb a part of the liquid.

With the carbon produced by the method for the material treatment of raw materials having the structure of the three-dimensional arrangement of carbon nanoparticles and a very large surface area, a maximum capacitance is achieved with a minimum volume and weight, since in particular the surface of the electrodes 43 determines the value of the capacitance. In addition, the electrodes 43 formed from the carbon are chemically inert with respect to the electrolyte and have a high temperature stability.

Phosphoric acid, H₃PO₄ for short, saturated with sodium hydroxide, NaOH for short, can be used as the electrolyte. Sodium hydroxide is also added. 10 ml of phosphoric acid are added to six beads of sodium hydroxide in order to increase the electric conductivity.

The separator 44 serving to prevent a short circuit within the storage device 40 is impregnated with the electrolyte. The strength and the density of the separator 44 determine the voltage and the automatic discharge of the supercapacitor. A lint-free paper layer in the form of pure cellulose with a thickness of about 0.1 mm can serve as separator 44. The paper layer can be formed as a double layer.

The storage device 40 thus produced can be charged as an energy cell at 0.5 V. In this case, the energy is absorbed in the shortest possible time without a change in temperature and can also be quickly released again without a change in temperature. With the characteristics of the supercapacitor, a voltage of 0.5 V to more than 10 V can be achieved. The storage device 40 can be scaled to different sizes. An electric energy store can be formed from a plurality of storage devices 40.

A storage device 40 having a total weight of 2 g contains 0.3 g of carbon and is fully charged at a voltage of 5.24 V within a period of 1 s. The storage device has an electric capacity of 140 mF. The current intensity is therefore 0.73 A. The size of such a storage device 40 is scalable.

In a comparison with conventional materials for the production of supercapacitors with activated carbon from coconut fibres as electrodes and otherwise identical parameters and dimensions, the electric capacity is only 71 mF. The storage device produced with the activated carbon also discharges many times faster. The differences are mainly due to the different values of the BET surface of the activated carbon compared to the amorphous carbon produced by the method for the material treatment of raw materials with the structure of the three-dimensional arrangement of carbon nanoparticles. The amorphous carbon also has a multiple higher heat resistance.

According to alternative embodiments, the collectors 42 are formed from a graphite foil which is less susceptible to acid and accordingly permits a higher flexibility in the choice of electrolyte, and an aluminium foil.

In order to increase the possible voltage of the storage device 40, the carbon can be purified, in particular in an acid bath, before processing, or heated to up to 800° C. for activation, for example in a microwave. Instead of the sodium water glass, polyurethane, casein or acetone with white glue can be used as binder. The electrodes 43 can each be in folded form in order to increase the contact surface.

Water-based solutions, such as zinc and sodium sulphates, and organic solutions, such as ethyl acetate, are used as electrolyte. The electrolyte can be prepared on a water basis with sodium sulphate, Na₂SO₄ for short, and preferably activated, for example, in a microwave. A redox electrolyte can likewise be used as the electrolyte.

A synthetic material, in particular a thin polypropylene membrane with very fine pores or a glass fibre fabric, can be used as separator 44.

### LIST OF REFERENCE NUMERALS

1 device for material treatment
2 heating system
3 distillation unit
4 reaction unit
5 bottom element of the heating system 2
6 support element
7 head element of the heating system 2
8 jacket element of the heating system 2
9 enclosure
10, 10a, 10b exhaust gas port
11, 11a, 11b exhaust gas line
11-1 connecting element of the exhaust gas line 11, 11a
12 cooling section of the distillation unit 3
12-1 air guide housing
12-2 fan
13 oil tank
14-1 extraction device
14-2 oil conveying device
15 control device
16a, 16b heating element
17 hood
18 connecting element of the exhaust gas line 11, 11a
19 tube connection
20 heating device
21 wall of the reaction unit 4
22 cover element
23 screen element
24 bottom plate
25 centring device for jacket element 8
26 jacket surface of centring device 25
27 sealing surface of centring device 25
28 support element for reaction unit 4
29 terminal surface
30 branch
31 opening point
32 inlet port of the oil tank 13
33 first outlet port of the oil tank 13
34 second outlet port of the oil tank 13
35 float
36 connecting port of the cover element 22
40 storage device
41 housing
42 collector
43 electrode
44 separator
B movement direction of the heating system 2
H height
T1, T2, T3 temperature sensor

1.-24. (canceled)

25. A carbon produced by a process for the material treatment of carbon-containing raw materials, the process comprising:
heating a reaction unit charged with carbonaceous raw materials and arranged in a closed heating system and starting a charring and distillation process, wherein the charring and distillation process takes place by targeted heating at a substantially constant temperature within the reaction unit;
discharging gases generated from the reaction unit into a distillation unit through an exhaust line formed between the reaction unit and the distillation unit and determining the temperature of the gas flowing through the exhaust line;
cooling and condensing the gases in the distillation unit, whereby the temperature of the gases is controlled by a forced cooling of a cooling section of the distillation unit via a heat output dissipated by the gases and the temperature of the gases is in a range from 95° C. to 125° C.;
extracting non-condensable gases, whereby a negative pressure to the environment is generated within the reaction unit and oxygen is removed from the reaction unit;
cooling the reaction unit, wherein, during the charring and distillation process and/or during the process of cooling the reaction unit, a gaseous flushing medium is flowed into the reaction unit; and
removing the final products from the reaction unit, wherein the gaseous flushing medium is applied to the reaction unit,
the carbon being for medical use and having:
a structure of a three-dimensional arrangement of carbon nanoparticles as agglomerates, wherein the carbon is amorphous and the carbon nanoparticles are cross-linked without long-range order, do not have a large-scale graphitic arrangement or structural similarity to graphene and are not arranged as nanotubes, and
a BET surface area greater than 2,500 m²/g BET.

26. The carbon according to claim 25, wherein the medical use is at least one of:
hemoperfusion/adsorption;
topical application on skin;
treatment of wounds and wound surfaces;
oral administration as an antidote in case of poisoning;
a carrier for antibiotics; or
a coating on implants.

27. The carbon according to claim 25, wherein the carbon has a BET surface area greater than 3,500 m²/g BET.

28. The carbon according to claim 25, wherein the carbon has a BET surface area greater than 4,000 m²/g BET.

29. The carbon according to claim 25, wherein the carbon has a BET surface area from 2,500 m²/g BET to 9,500 m²/g BET.

30. The carbon according to claim 25, wherein the carbon has a BET surface area from 4,200 m²/g BET to 4,500 m²/g BET.

31. A method comprising:
employing a thermal, refractory, or radiation-resistant insulating material, the insulating material including carbon, the carbon:
having a structure of a three-dimensional arrangement of carbon nanoparticles as agglomerates, wherein the carbon is amorphous and the carbon nanoparticles are cross-linked without long-range order, do not have a large-scale graphitic arrangement or structural similarity to graphene and are not arranged as nanotubes;
having a BET surface area greater than 2,500 m²/g; and
being produced by a process comprising:
heating a reaction unit charged with carbonaceous raw materials and arranged in a closed heating system and starting a charring and distillation process, wherein the charring and distillation process takes place by targeted heating at a substantially constant temperature within the reaction unit;
discharging gases generated from the reaction unit into a distillation unit through an exhaust line formed between the reaction unit and the distillation unit and determining the temperature of the gas flowing through the exhaust line;
cooling and condensing the gases in the distillation unit, whereby the temperature of the gases is controlled by a forced cooling of a cooling section of the distillation unit via a heat output dissipated by the gases and the temperature of the gases is in a range from 95° C. to 125° C.;
extracting non-condensable gases, whereby a negative pressure to the environment is generated within the reaction unit and oxygen is removed from the reaction unit;
cooling the reaction unit, wherein, during the charring and distillation process and/or during the process of cooling the reaction unit, a gaseous flushing medium is flowed into the reaction unit; and
removing the final products from the reaction unit, wherein the gaseous flushing medium is applied to the reaction unit.

32. The method of claim 31, wherein employing the insulating material comprises using the insulating material as one of:
a component of a heat shield in rockets or space gliders;
a component of an enclosure of a power station or nuclear reactor; or
a component of a wall of a house.

33. The method according to claim 31, wherein the carbon of the insulating material employed has a BET surface area from 2,500 m²/g BET to 9,500 m²/g BET.

34. The carbon according to claim 31, wherein the carbon of the insulating material employed has a BET surface area from 4,200 m²/g BET to 4,500 m²/g BET.

35. A method comprising:
employing a filter element, the filter element including carbon, the carbon:
having a structure of a three-dimensional arrangement of carbon nanoparticles as agglomerates, wherein the carbon is amorphous and the carbon nanoparticles are cross-linked without long-range order, do not have a large-scale graphitic arrangement or structural similarity to graphene and are not arranged as nanotubes;
having a BET surface area greater than 2,500 m²/g BET; and
being produced by a process comprising:
heating a reaction unit charged with carbonaceous raw materials and arranged in a closed heating system and starting a charring and distillation process, wherein the charring and distillation process takes place by targeted heating at a substantially constant temperature within the reaction unit;
discharging gases generated from the reaction unit into a distillation unit through an exhaust line formed between the reaction unit and the distillation unit and determining the temperature of the gas flowing through the exhaust line;
cooling and condensing the gases in the distillation unit, whereby the temperature of the gases is controlled by a forced cooling of a cooling section of the distillation unit via a heat output dissipated by the gases and the temperature of the gases is in a range from 95° C. to 125° C.;
extracting non-condensable gases, whereby a negative pressure to the environment is generated within the reaction unit and oxygen is removed from the reaction unit;
cooling the reaction unit, wherein, during the charring and distillation process and/or during the process of cooling the reaction unit, a gaseous flushing medium is flowed into the reaction unit; and
removing the final products from the reaction unit, wherein the gaseous flushing medium is applied to the reaction unit.

36. The method of claim 35, wherein employing the filter element comprises using the filter element as one of:
an air filter element;
a water filter element;
a filter for filtering out salt, oil, petrol, iodine or acid from water;
a component of an air-conditioning system; a component of a breathing mask; or
a component of an exhaust gas device.

37. The method according to claim 35, wherein the carbon of the filter element employed has a BET surface area from 2,500 m²/g BET to 9,500 m²/g BET.

38. The carbon according to claim 35, wherein the carbon of the filter element employed has a BET surface area from 4,200 m²/g BET to 4,500 m²/g BET.

39. A method comprising:
employing a storage element, the storage element including carbon, the carbon:
having a structure of a three-dimensional arrangement of carbon nanoparticles as agglomerates, wherein the carbon is amorphous and the carbon nanoparticles are cross-linked without long-range order, do not have a large-scale graphitic arrangement or structural similarity to graphene and are not arranged as nanotubes;
having a BET surface area greater than 2,500 m²/g BET; and
being produced by a process comprising:
heating a reaction unit charged with carbonaceous raw materials and arranged in a closed heating system and starting a charring and distillation process, wherein the charring and distillation process takes place by targeted heating at a substantially constant temperature within the reaction unit;
discharging gases generated from the reaction unit into a distillation unit through an exhaust line formed between the reaction unit and the distillation unit and determining the temperature of the gas flowing through the exhaust line;
cooling and condensing the gases in the distillation unit, whereby the temperature of the gases is controlled by a forced cooling of a cooling section of the distillation unit via a heat output dissipated by the gases and the temperature of the gases is in a range from 95° C. to 125° C.;
extracting non-condensable gases, whereby a negative pressure to the environment is generated within the reaction unit and oxygen is removed from the reaction unit;
cooling the reaction unit, wherein, during the charring and distillation process and/or during the process of cooling the reaction unit, a gaseous flushing medium is flowed into the reaction unit; and
removing the final products from the reaction unit, wherein the gaseous flushing medium is applied to the reaction unit.

40. The method of claim 39, wherein employing the storage element comprises using the storage element as one of:
a component of an electric energy store;
a component of a battery;
a component of a capacitor; or
a component of a data storage.

41. The method of claim 39, wherein employing the storage element comprises using the storage material as a battery of a motor vehicle

42. The method according to claim 39, wherein the carbon of the storage element employed has a BET surface area from 2,500 m²/g BET to 9,500 m²/g BET.

43. The carbon according to claim 39, wherein the carbon of the storage element employed has a BET surface area from 4,200 m²/g BET to 4,500 m²/g BET.

44. An electric energy storage device in the form of a double-layer capacitor, comprising:
a symmetrical structure including:
a housing and a center separator with an electrolyte within the housing;
a pair of collectors positioned on opposite sides of the collector; and
a pair of electrodes in the form of a carbon layers, the electrodes positioned between the collectors and the housing, the carbon layers including carbon:
having a structure of a three-dimensional arrangement of carbon nanoparticles as agglomerates, wherein the carbon is amorphous and the carbon nanoparticles are cross-linked without long-range order, do not have a large-scale graphitic arrangement or structural similarity to graphene and are not arranged as nanotubes;
having a BET surface area greater than 2,500 m²/g BET; and
being produced by a process comprising:
heating a reaction unit charged with carbonaceous raw materials and arranged in a closed heating system and starting a charring and distillation process, wherein the charring and distillation process takes place by targeted heating at a substantially constant temperature within the reaction unit;
discharging gases generated from the reaction unit into a distillation unit through an exhaust line formed between the reaction unit and the distillation unit and determining the temperature of the gas flowing through the exhaust line;
cooling and condensing the gases in the distillation unit, whereby the temperature of the gases is controlled by a forced cooling of a cooling section of the distillation unit via a heat output dissipated by the gases and the temperature of the gases is in a range from 95° C. to 125° C.;
extracting non-condensable gases, whereby a negative pressure to the environment is generated within the reaction unit and oxygen is removed from the reaction unit;
cooling the reaction unit, wherein, during the charring and distillation process and/or during the process of cooling the reaction unit, a gaseous flushing medium is flowed into the reaction unit; and
removing the final products from the reaction unit, wherein the gaseous flushing medium is applied to the reaction unit.

45. A method comprising:
employing a carbon-containing agricultural product, wherein the carbon serves as a water reservoir and nutrient reservoir, the carbon:
having a structure of a three-dimensional arrangement of carbon nanoparticles as agglomerates, wherein the carbon is amorphous and the carbon nanoparticles are cross-linked without long-range order, do not have a large-scale graphitic arrangement or structural similarity to graphene and are not arranged as nanotubes;
having a BET surface area greater than 2,500 m²/g BET; and
being produced by a process comprising:
heating a reaction unit charged with carbonaceous raw materials and arranged in a closed heating system and starting a charring and distillation process, wherein the charring and distillation process takes place by targeted heating at a substantially constant temperature within the reaction unit;
discharging gases generated from the reaction unit into a distillation unit through an exhaust line formed between the reaction unit and the distillation unit and determining the temperature of the gas flowing through the exhaust line;
cooling and condensing the gases in the distillation unit, whereby the temperature of the gases is controlled by a forced cooling of a cooling section of the distillation unit via a heat output dissipated by the gases and the temperature of the gases is in a range from 95° C. to 125° C.;
extracting non-condensable gases, whereby a negative pressure to the environment is generated within the reaction unit and oxygen is removed from the reaction unit;
cooling the reaction unit, wherein, during the charring and distillation process and/or during the process of cooling the reaction unit, a gaseous flushing medium is flowed into the reaction unit; and
removing the final products from the reaction unit, wherein the gaseous flushing medium is applied to the reaction unit.

46. The method of claim 45, wherein employing the agricultural product comprises using the agricultural product for one of:
enriching upper soil layers;
producing vegetable foodstuffs; or
stabilizing natural stalk in cereal production.

47. The method of claim 45, wherein employing the agricultural product comprises introducing the agricultural product into soil over a large area in one or more layers.

48. The method of claim 45, wherein employing the agricultural product comprises introducing the agricultural product into soil over a large area at a depth of approximately 20 cm to 30 cm.
